(19) United States
(12) Patent Application Publication
(10) Pub. No.: US 2013/0116186 A1
(43) Pub. Date: May 9, 2013

(54) LUBRICIN INJECTIONS TO MAINTAIN CARTILAGE HEALTH

(71) Applicant: Rhode Island Hospital, A Lifespan Partner, Providence, RI (US)

(72) Inventor: Gregory Jay, Norfolk, MA (US)

(73) Assignee: Rhode Island Hospital, A Lifespan Partner, Providence, RI (US)

(21) Appl. No.: 13/644,945

(22) Filed: Oct. 4, 2012

Related U.S. Application Data
(60) Provisional application No. 61/542,943, filed on Oct. 4, 2011.

(51) Int. Cl. A61K 38/39

(52) U.S. Cl. CPC A61K 38/39
USPC 514/17.1

(57) ABSTRACT

Disclosed are novel methods of maintaining articular cartilage health in an atraumatic, non-diseased joint of a mammal, either human or non-human. The methods involve injecting into the joint capsule of the mammal an amount of a lubricin polypeptide to prevent shear-induced chondrocyte apoptosis and to maintain a lubricin-expressing phenotype of chondrocytes.

### CROSS-REFERENCE TO RELATED APPLICATIONS

This application claims priority to and the benefit of U.S. Provisional Patent Application No. 61/542,943, filed Oct. 4, 2011, the contents of which are incorporated by reference herein.

### STATEMENT REGARDING FEDERALLY SPONSORED RESEARCH OR DEVELOPMENT

This invention was made with government support under Grant No. ROIAR50180 awarded by the National Institutes of Health. The government has certain rights in this invention.

### BACKGROUND OF THE INVENTION

Lubricin is a well-known glycoprotein which holds great promise in a variety of preventative and therapeutic medical procedures. Lubricin, also known as superficial zone protein and PRG4, has been suggested for use in treatment of injured joints and in treatment of degenerative joint disease such as osteoarthritis by intra-articular injection. It has been suggested for use during or post-surgery to inhibit formation of tissue adhesions. It can be used as a lubricating and bacteria-repelling coating for devices intended for prolonged contact with the body such as implantable devices, catheters, and ophthalmic lenses. It also has been suggested to treat interstitial cystitis, oral ulcerations, and dry mouth, and is useful as an ingredient in preparations which reduce the coefficient of friction between bearing tissue surfaces, e.g., to lubricate tendons, as a vaginal lubricant, and as an ocular boundary lubricant in eye drops and contact lens treatments, e.g., for treatment of dry eye, Sjogren's syndrome, and other ocular surface disorders.

It is well know that lubricin lubricates bearing surfaces via a boundary lubrication mechanism. The most studied surface, cartilage surface, under load and fully pressurized, has been shown to interact through semi flattened asperity (surface micro-irregularity) contacts. Boundary lubrication by lubricin prevents a stick-slip phenomena which reduces mechanical strain which superficial zone chondrocytes would experience.

Blunt trauma has been described as leading to chondrocyte apoptosis. Chondrocyte apoptosis has been observed in osteoarthritic cartilage prior to cartilage breakdown and fibrillation. Cell death affects cartilage metabolism and contributes to joint failure by decreasing the structural integrity of cartilage. Patients with Camptodactyly-Arthropathy-CoxaVara-Pericarditis syndrome (CACP), who have no ability to produce lubricin, develop precocious joint failure due to its absence; however, other synovial fluid constituents appear normal in CACP patients. In an in vitro study, CACP synovial fluid was unable to lubricate a latex-on-glass bearing system. Also, the joints of lubricin-null mice exhibit higher coefficients of friction when tested in an ex vivo pendulum apparatus, and experience higher degrees of wear.

Articular joints employ lubricin as a boundary lubrication to repel surface asperities and protect the superficial zone of the cartilage. Boundary lubrication provides effective chondroprotection by decreasing friction between surface asperities that are pressurized together during surface-to-surface contact. In the absence of this lubrication, superficial zone chondrocytes are very likely deformed much as is the superficial zone deformed during a stick-slip phenomena as a result of elevated friction.

### SUMMARY OF THE INVENTION

Prior to this invention, it was understood that trauma to a joint induced chondrocyte apoptosis. However, prior to Applicants' invention, chondrocyte apoptosis had not been observed as a result of sheer strain or friction in the joint. Applicants have now observed that superficial zone chondrocytes become apoptotic immediately following the frictional response in a poorly lubricated cartilage weight-bearing surface. Superficial zone chondrocytes die as a direct result of friction, e.g., as the result of shear strain. More particularly, it has been discovered that a breakdown of boundary lubrication on cartilage surface relates directly to chondrocyte apoptosis and destruction of extracellular matrix. These cells die typically within twenty-four hours.

It also has been discovered that application of an exogenous lubricin, e.g., recombinant lubricin, to a cartilage surface by direct injection into a joint capsule has the unexpected effect of inhibiting chondrocytes from entering an apoptotic pathway and therefore retaining a healthy, homeostatic phenotype wherein the chondrocytes maintain their structure within the cartilage and secrete lubricin. It is believed that lubricin has this unexpected effect of preventing chondrocyte apoptosis because the application of lubricin prevents toxic deformation of chondrocytes, and not because lubricin interacts with a receptor or exhibits a classical drug effect. In fact, it is believed that lubricin's lubricating effect is the result of the physical and mechanical properties of the protein alone, and is not a result of a traditional ligand/receptor interaction exhibited by many proteins.

Through testing using partly purified human lubricin, human synovial fluid from healthy patients, and patients with CACP (which essentially is compositionally identical to healthy synovial fluid except for the absence of lubricin), it has been demonstrated that chondrocyte apoptosis inversely correlates with the effectiveness of boundary lubrication.

Accordingly, in one aspect, the invention provides a novel method of maintaining articular cartilage health in an atraumatic, non-diseased joint of a mammal. The method involves injecting into the joint capsule of the mammal, including a human, an amount of a lubricin polypeptide sufficient to substantially prevent shear-induced chondrocyte apoptosis and to maintain a lubricin-expressing phenotype of chondrocytes. The joint may be, for example, a knee, hip, elbow, wrist, ankle, shoulder, finger or toe joint, or other cartilaginous joint. In a further embodiment, the joint is a weight bearing joint. The non-human mammal may be, for example, a horse, a dog, or a cat. In this aspect, lubricin is used as a joint maintenance adjuvant or supplement, or a prophylaxis against the development of joint pain and deterioration of function. For example, it may be suitable for injection into the knee or hip or other joint of persons approaching or passing into old age, persons at risk of osteoarthritis, e.g., having a history of osteoarthritis in their genetic background, or having suffered a previous joint trauma, now healed, which predisposes the person to osteoarthritis later in life. The method is well suited for treatment of persons preparing for strenuous or dangerous joint-stressing activities, such as athletic activities. One effect of such treatments is to enable cartilage to repopulate with chondrocytes; thus, treatment of lubricin maintains and may actually improve cartilage health, essentially triggering a feedback mechanism whereby administration of exogenous lubricin leads to improvements in endogenous lubricin expression.

In other embodiments, the method of the invention is well-suited as a treatment for idiopathic or asymptomatic joint pain, i.e., where the pain occurs in an atraumatic, non-diseased joint where no clinical diagnosis of cartilage damage, such as osteoarthritis or other diagnosable joint degeneration or trauma, can be made. In such patients, idiopathic joint pain may be intermittent or transient and therefore resolve on its own. In such patients, the joint does not exhibit symptoms leading to diagnosis of cartilage damage. Such patients are well-suited for treatment according to the methods of the invention. Accordingly, in another aspect, the invention provides a novel method of maintaining articular cartilage health in an atraumatic, non-diseased joint of a mammal experiencing idiopathic or transient joint pain. The method involves injecting into the joint capsule of the mammal, including a human, an amount of a lubricin polypeptide sufficient to substantially prevent shear-induced chondrocyte apoptosis and to maintain a lubricin-expressing phenotype of chondrocytes. The joint may be, for example, a knee, hip, elbow, wrist, ankle, shoulder, finger or toe joint, or other cartilaginous joint.

Many people have chronic joint pain characterized as idiopathic synovitis or unrelated to diagnosed joint disease. Some receive periodic injections of hyaluronic acid preparations into the joint for relief of pain. Recently, it was reported by Briem et al., (2009) J. Orthop. Res., 27:1420-5, that the pain relieving effect of administration of hyaluronic acid may actually accelerate joint deterioration and exacerbate cartilage pathology because the patient uses the joint more frequently and freely as his joint pain is decreased. Accordingly, in another aspect, the inventive method is practiced on a mammal previously injected with hyaluronic acid or other form of hyaluron as a treatment for idiopathic joint pain so as to prospectively inhibit chondrocyte apoptosis and to restore a lubricin-expressing phenotype in chondrocytes. In one embodiment, the method may also be practiced on a patient who is receiving hyaluronic acid or other form of hyaluron as a treatment for idiopathic joint pain so as to prospectively inhibit chondrocyte apoptosis and to restore a lubricin-expressing phenotype in chondrocytes. For example, according to one embodiment, a patient may receive administration of lubricin in connection with receiving administration of hyaluronic acid or other form of hyaluron to treat idiopathic joint pain. The administration of hyaluronic acid or other form of hyaluron may occur before or after the administration of lubricin. In one embodiment, lubricin is co-administered with hyaluronic acid or another form of hyaluron. Administration is, for example, by injection into the joint capsule. The joint may be, for example, a knee, hip, elbow, wrist, ankle, shoulder, finger or toe joint.

In one preferred embodiment, the inventive method comprises injecting an amount of a lubricin polypeptide sufficient to prevent separation of fibronectin and collagenous extracellular cartilage matrix components from chondrocyte surface integrins. In a further preferred embodiment, the inventive method comprises injecting a recombinant form of a human lubricin polypeptide which contains multiple repeats of an amino acid sequence which is at least 50% identical to KEPAPTT and comprises O-linked β-(1-3)-Gal-GalNac moieties, optionally but preferably capped with NeuAc. In a further preferred embodiment, the inventive method comprises injecting an amount of a lubricin polypeptide sufficient to maintain a close packed field of lubricin molecules over weight bearing cartilage surface within the joint capsule. In yet another preferred embodiment, the inventive method comprises injecting an amount of a lubricin polypeptide sufficient to establish a lubricin concentration in the synovial fluid within the capsule preferably of between 100 μg/mL and 500 μg/mL, preferably at least greater than 250 μg/mL.

In another aspect, the invention is embodied as a lubricin polypeptide for the manufacture of a medicament for the inhibition of apoptosis of chondrocytes in an articular cartilage of a mammal. In yet another aspect, the invention is embodied as a recombinant lubricin polypeptide for the manufacture of a medicament for stimulation of chondrocytes disposed in the superficial zone of articular cartilage of a mammal to produce endogenous lubricin. In another aspect, the invention is embodied as a lubricin polypeptide for the manufacture of a medicament for maintaining articular cartilage health in a mammalian patient experiencing idiopathic or transient joint pain in an atraumatic, non-diseased joint. In yet another aspect, the invention is embodied as a lubricin polypeptide for the manufacture of a medicament for maintaining articular cartilage health in a non-diseased, atraumatic joint of a mammal.

### BRIEF DESCRIPTION OF THE FIGURES

FIG. 1 shows three photographs demonstrating the results of activated caspase-3 staining in bovine cartilage samples. Caspase-3 activity is an indicator chondrocyte apoptosis. The left pane shows the unloaded control; the center pane shows cartilage samples lubricated with phosphate buffered saline (PBS); the right pane shows cartilage samples lubricated with human synoviocyte lubricin (HSL). As the images indicate, HSL and unloaded control samples showed little to no activated caspase-3 cellular activity, whereas caspase-3 activity, indicated by the arrows pointing to positively stained cells, was present in the PBS treated cartilage.

FIG. 2 is a bar graph showing the static (top) and kinetic (bottom) coefficients of friction for each of the PBS, CACP-SF, and HSL lubricant groups. Error bars represent standard error.

FIG. 3 is the amino acid sequence for full-length human lubricin.

### DETAILED DESCRIPTION

### Lubricin

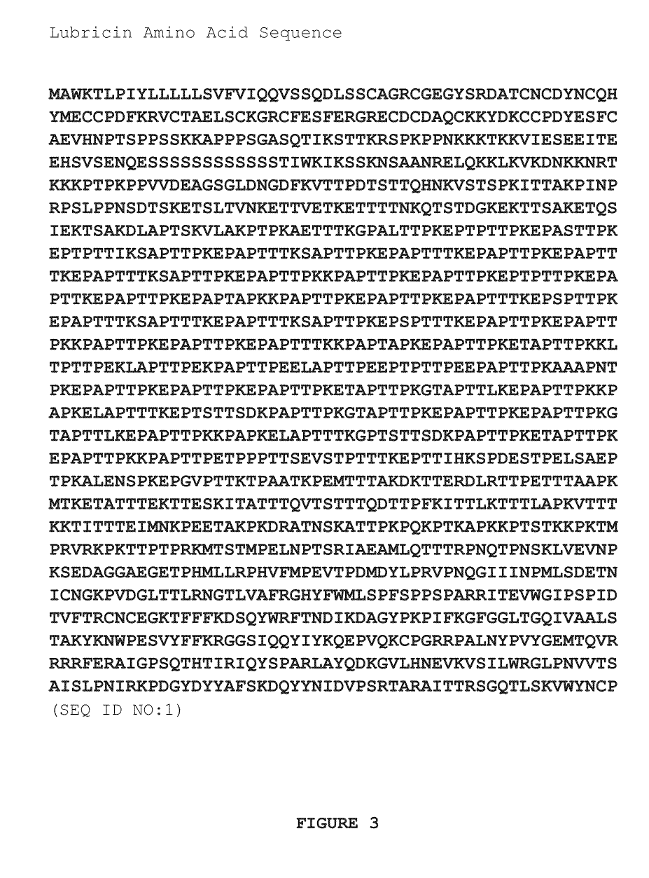

Lubricin is a lubricating polypeptide, which in humans is expressed from the megakaryocyte stimulating factor (MSF) gene, also known as PRG4. The amino acid sequence of full-length human lubricin is shown in FIG. 3 and can be obtained as NCBI Accession Numbers AK131434 and U70136. Its natural form in all mammals investigated contains multiple repeats of an amino acid sequence which is at least 50% identical to the sequence KEPAPTT (SEQ ID NO:2). Natural lubricins typically comprise multiple redundant forms of this repeat, but typically always include proline and threonine residues, with at least one threonine being glycosylated in some, most, or all repeats. The threonine anchored O-linked sugar side chains are critical for lubricin's boundary lubricating function. The side chain moiety typically is a β-(1-3)-Gal-GalNac moiety. Optionally, the β-(1-3)-Gal-GalNac is capped with sialic or N-acetylneuraminic acid or NeuAc. The polypeptide also typically comprises N-linked oligosaccharides. Lubricin is polydisperse in that individual lubricin molecules differ in their degree and pattern of glycosylation.

The gene encoding naturally-occurring full length lubricin contains 12 exons. The naturally-occurring MSF gene product contains 1404 amino acids with multiple polypeptide sequence homologies to vitronectin including hemopexin-like and somatomedin-like regions. Centrally-located exon 6 contains 940 residues. Exon 6 encodes the KEPAPPT repeat rich, O-glycosylated mucin domain (amino acids 200-1140 of SEQ ID NO:1) which provides boundary lubrication of articular cartilage. The full-length sequence of human lubricin is set forth in FIG. 3. The MSF gene product is extensively characterized, for example, in WO 00/64930, the disclosure of which is incorporated by reference herein.

The amino acid sequence of the protein backbone of lubricin polypeptides may differ depending on alternative splicing of exons of the human MSF gene. Because lubricin serves a fundamentally mechanical function, its fine tertiary structure is less critical than proteins such as cytokines or antibodies where subtle stereochemistry governs binding to receptors.

The amino acid sequence of the backbone of purified, synthetic, or recombinant lubricin useful in this invention may be at least 50% identical to the amino acid sequence of a naturally-occurring human lubricin, and possess at least 50% of the lubricating activity of a naturally-occurring lubricin. In other embodiments, the amino acid sequence of the backbone of purified, synthetic, or recombinant lubricin useful in this invention may be at least 55%, 60%, 65%, 70%, 75%. 80%. 85%, 90%, 95%, or 98% identical to the amino acid sequence of a naturally-occurring human lubricin. In other embodiments, the amino acid sequence of the backbone of purified, synthetic, or recombinant lubricin useful in this invention may possess at least 55%, 60%, 65%, 70%, 75%. 80%. 85%, 90%, 95%, or 98% of the lubricating activity of a naturally-occurring lubricin. A standard friction apparatus can be used to measure lubricating activity according to methods described by Jay et al., (1992), Conn. Tiss. Res., 28:71-88 or Jay et al., (1998), J. Biomed. Mater. Res., 40:414-418, and described in, for example, WO 00/64930, the disclosure of which is incorporated by reference herein.

Lubricin polypeptides include lubricating fragments, i.e., polypeptides which are not full length lubricin, but which possess lubricating activity of lubricin and sequence identity with lubricin. For example, lubricating fragments may possess at least 50%, 55%, 60%, 65%, 70%, 75%, 80%, 85%, 90%, 95%, 97%, 98%, or 99% of the lubricating activity of a naturally-occurring lubricin. Lubricating fragments may be at least 50%, 55%, 60%, 65%, 70%, 75%, 80%, 85%, 90%, 95%, 97%, 98%, or 99% identical to the amino acid sequence of a naturally-occurring human lubricin. Lubricating fragments of human lubricin may be identified by the presence of multiple repeats of the repeating amino acid sequence KEPAPPT (SEQ ID NO:2). Lubricating fragments of lubricin from other mammalian species may be identified by multiple repeats of a sequence that is at least 50% identical to the sequence KEPAPTT (SEQ ID NO:2).

Lubricin polypeptides include naturally occurring, as well as synthetic or recombinant isoforms and variants. For example, a lubricin polypeptide may contain amino acids 67-106 and 200-1140 of SEQ ID NO:1 and multiple O-linked oligosaccharide, but lack amino acids 26-66 of SEQ ID NO:1. Other variants may be used. For example, the polypeptide may lack amino acids 107-156 of SEQ ID NO:1 or amino acids 107-199 of SEQ ID NO:1. For example, the polypeptide contains amino acids 1-25, 67-106, and 200-1404 of SEQ ID NO:1. Alternatively, the polypeptide contains amino acids 67-106 and 200-1140 of SEQ ID NO:1 and O-linked oligosaccharides, but lacks amino acids 107-156 of SEQ ID NO:1. In another variation, the lubricating polypeptide contains amino acids 67-106 and 200-1140 of SEQ ID NO:1 and O-linked oligosaccharides, but lacks amino acids 157-199 of SEQ ID NO:1. For example, the polypeptide contains amino acids 1-156 and 200-1404 of SEQ ID NO:1 (lacking amino acids 157-199 of SEQ ID NO:1) or the polypeptide contains amino acids 1-106 and 200-1404 (lacking amino acids 107-199 of SEQ ID NO:1). The amino acid sequences are contiguous to one another or may be separated by intervening unrelated sequences. Preferably, a lubricin polypeptide contains residues encoded by exon 1 (amino acids 1-24 of SEQ ID NO:1), exon 3 (amino acids 67-104), and exons 6-12 (amino acids 200-1404) of the MSF gene.

A number of naturally-occurring human lubricin isoforms have been identified and isolated. For example, a human lubricin isoform was purified from synovial fluid and found to contain amino acids encoded by exons 1, 3, and 6-12 of the MSF gene (lacking exons 2, 4, or 5).

Lubricins may be purified from naturally-occurring synovial fluids or synthetically produced by recombinant DNA technology. For teaching on how to make various forms of lubricin, including various truncated and mutated versions, see, for example, U.S. Published Patent Application Nos. 2004/0229804, 2006/0240037, and 2007/0191268, the disclosures of which are incorporated herein by reference.

### Candidates for Prophylactic Lubricin Administration

It is known to use lubricin as an injectable device, either alone or together with hyaluronic acid products, to treat traumatized knees and hips, and other joints diagnosed as suffering from arthritis or other degenerative joint disease such as osteoarthritis, rheumatoid arthritis, or psoriatic arthritis. However, in accordance with this invention, the use of lubricin is expanded to malfunctioning, but asymptomatic joints, or “atraumatic joints,” that is, joints at risk of deterioration in function, and healthy joints that will or may be subjected to hard use or potential trauma, which in turn may lead to latent or overt joint deterioration. The term “atraumatic,” as used herein, means that the joint has not been diagnosed as suffering from a physical injury such as meniscus tear, ACS injury, or cartilage loss or damage, but includes cases of subclinical joint trauma, and cases of idiopathic joint pain or asymptomatic joint pain.

Further, in accordance with this invention, lubricin is indicated for administration to joints wherein a patient experiences intermittent or transient pain (i.e., resolves on its own) and/or idiopathic pain in the joint, but the joint is not clinically diagnosable as having any condition relating to cartilage degeneration or damage.

Injection of lubricin into a joint is contraindicated in situations where joint tissue must heal, e.g., locations of repair with resorbable sutures or tissue adhesives, as lubricin exhibits potent anti-adhesive properties that may interfere with natural healing of wounds and repair of surgical incisions and other tissue separations. The use of lubricin in these contexts may require additional tissue engineering strategies to promote healing such as stem cells in situ. However, intervention with lubricin in medical conditions where cartilage tissue is at risk of degradation from aging, stress, overuse, poor gait, strain, or anatomical irregularity can result in preservation of chondrocyte density in articular cartilage and maintenance or improvement in joint health.

Patients with subclinical joint trauma or those who anticipate experiencing sub-clinical joint trauma are candidates for this therapy. This could include patients who will likely cause an overuse syndrome of a knee, for example. Thus the soccer player, football player, and the marathon runner may be candidates. Any athlete involved in an activity likely to cause sub-clinical joint trauma is a candidate. Likewise patients with an episodic case of gout or pseudo-gout are candidates as well. These patients have in common the manifestations of acquired joint synovitis, in the absence of established joint disease or arthropathy. Given the high likelihood of reactive synovitis as a direct result of the above avocations, these individuals would be candidates for the preventive supplementation practice using a lubricin polypeptide. These individuals would receive a joint injection of lubricin to help enhance the protective coating of lubricin on the articular surface which synovitis would otherwise catabolize and render ineffective. Thus any individual about to experience episodic joint overuse would prophylax with lubricin intra-articular injection to re-establish joint homeostasis in an anticipatory way. Joints such as the knee, hip, elbow, shoulder, wrist, ankle, finger or toe are joints that can be treated according to the invention.

Candidates for therapy according to the methods of invention also include patients who have received, are receiving, or will receive administration of hyaluronic acid or other hyaluronate to a joint capsule. As described above, administration of hyaluronic acid or other hyaluronate to a joint capsule relieves pain, but this pain relief allows the patient to use the joint more frequently and more freely, thereby exacerbating any underlying cartilage pathology. Accordingly, administration of lubricin to the joint capsule prospectively inhibits chondrocyte apoptosis and restores or maintains a lubricin-expressing phenotype in chondrocytes.

Candidates for therapy according to the methods of the invention also include non-human mammals such as dogs, cats, or horses. For example, animals who will experience sub-clinical joint trauma are candidates for this therapy. For example, a horse may be administered lubricin intra-articularly via injection in one or more knee joints or hip joints prior to a race or carrying a load so as to prospectively inhibit chondrocyte apoptosis and restore or maintain a lubricin-expressing phenotype in chondrocytes of the joints. For example, a dog, cat or horse, who demonstrates idiopathic and/or transient joint pain (i.e., the animal is limping or the animal was limping but the limp has resolved), but is asymptomatic for and not diagnosable as having any condition associated with cartilage degeneration or damage is a candidate for administration of lubricin into the joint capsule.

### Administration

According to the methods of the invention, a lubricin polypeptide is injected into the joint capsule of a patient indicated for treatment. The amount of lubricin polypeptide administered will depend on the size of the joint being treated. For example, a hip or knee joint would require a larger amount of lubricin that a finger or toe joint in order to provide the prophylactic effect taught herein. Further, the size of a joint may vary from one mammal to another. Accordingly, the amount of lubricin administered is tailored to each individual recipient according to standard methods. Standard methods for delivery of peptides are used. Such methods are well known to those of ordinary skill in the art.

Administration of lubricin according to methods of the invention includes injecting an amount of a lubricin polypeptide sufficient to maintain a close packed field of lubricin molecules over weight bearing cartilage surface within the joint capsule. Administration of lubricin according to methods of the invention also includes injecting an amount of a lubricin polypeptide sufficient to establish a bioavailable lubricin concentration in the synovial fluid (as opposed to lubricin bound to cartilage) within the capsule preferably of between 100 μg/mL and 500 μg/mL, preferably at least greater than 250 μg/mL, and most preferably between 250 μg/mL and 450 μg/mL.

In one embodiment, a lubricin polypeptide is delivered to the synovial cavity at a concentration in the range of 20-500 μg/mL in a volume of approximately 0.1-2 mL per injection, more preferably 1-2 mL. For example, 1 mL of a lubricin polypeptide at a concentration of 250 μg/mL is injected into a knee joint using a fine (e.g., 14-22 gauge, preferably 18-22 gauge) needle.

In another embodiment, a lubricin polypeptide is delivered to the synovial cavity at a concentration from as low as 20 μg/mL to as high as 8 mg/mL or even as high as 10 mg/mL in, for example, 2 mL of fluid. In another embodiment, a lubricin polypeptide is delivered to the synovial cavity of a joint in the amount of 4 mg/mL-5 mg/mL in, for example, 2 mL of fluid. 2 mL of fluid is generally the appropriate volume of fluid for a large joint such as the hip, knee, or shoulder. The volume of fluid would likely be lower for smaller joints, for example 0.1 mL-1 mL.

According to some embodiments of the invention, administration of a lubricin polypeptide occurs only once. For example, administration of a lubricin polypeptide in a patient in connection with a sporting event or other activity where the patient is at risk of incurring sub-clinical joint trauma would occur once before the event. This administration could occur, for example, ½ hour, 1 hour, 2 hours, or up to 12 hours before the event.

In other embodiments of the invention, administration of lubricin occurs multiple times or even indefinitely in order to maintain articular cartilage health in an atraumatic, non-diseased joint of a mammal, to substantially prevent shear-induced chondrocyte apoptosis, and to maintain a lubricin-expressing phenotype of chondrocytes. For example, administration of a lubricin polypeptide to a joint capsule occurs once per month. However, in some instances, administration to a joint capsule may occur once every two months, once every three months, once every 6 months, or annually. In most cases, administration may continue indefinitely.

### Example 1

### The Effect of Boundary Lubrication on Chondrocyte Apoptosis in an In Vitro Cartilage Bearing System

Full thickness cartilage plugs 6 mm and 12 mm in diameter were drilled from the femoral condyle of a bovine knee collected within 2 hours of sacrifice. Following harvest, the plugs were rinsed in cell culture media and cultured for 24 hours at 37° C. Samples were loaded in an EnduraTEC model 3200 ElectroForce (ELF) system (Bose Corporation, ElectroForce Systems Group, Eden Prairie, Minn., USA), the remaining cell culture media was rinsed off with 1×PBS and the test lubricant, either phosphate buffered saline (PBS, 1×, n=5), human synoviocyte lubricin in PBS at 250 μg/ml (HSL), or CACP synovial fluid (CACP-SF) was applied between the surfaces (PBS n=5, HSL n=3, CACP-SF n=5). CACP-SF had been previously recovered following diagnostic or therapeutic aspiration.

The plugs were compressed to 18% total cartilage thickness and held for 8 minutes to allow fluid depressurization. The bottom plug was rotated in torsion +2 revolutions and reset −2 revolutions for 12 continuous cycles. The plugs were placed in formalin immediately following testing. Control samples (n=3) were not loaded in the apparatus, but were placed in formalin at the time of testing.

All tests were performed between 48 and 72 hours of harvest. Coefficient of friction (COF) values were determined using the equilibrium load and the maximum torque seen at the start of rotation (static) or the average torque seen at the last 720 degrees of rotation (kinetic). Histological analysis was performed using an antibody specific for activated caspase-3, a marker of apoptosis. Statistical analysis was performed using a one way ANOVA with Holm-Sidak comparison.

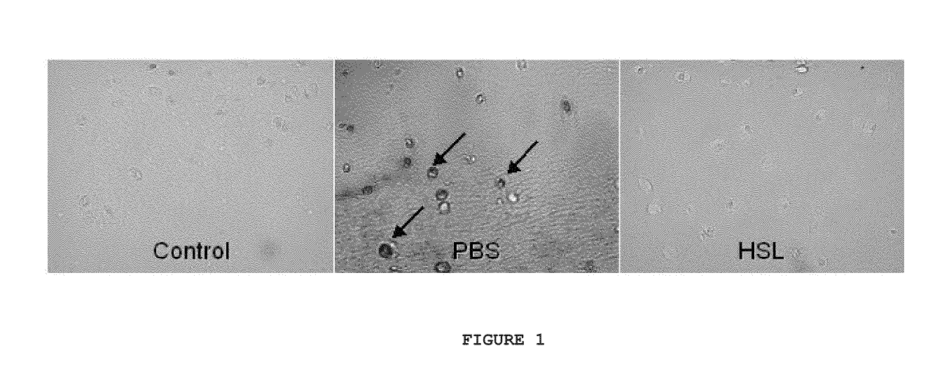

As shown in FIG. 1, an increase in activated caspase-3 staining was observed in the cells of plugs lubricated with PBS when compared to plugs lubricated with HSL or unloaded. Activated caspase-3 staining was observed in cartilage samples lubricated with PBS, indicating chondrocyte apoptosis. HSL and unloaded control samples showed little to no activated caspase-3 cellular activity, indicating no active apoptosis.

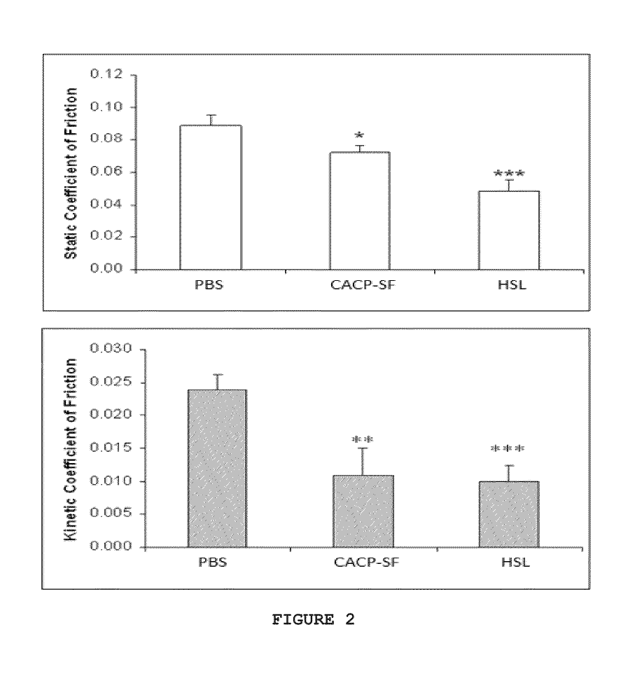

In a separate test, a set of cartilage plugs were tested using CACP-SF then immediately re-tested using a mixture of CACP-SF and HSL to observe any changes in coefficient of friction. The results are shown in FIG. 2. The static coefficient of friction decreased in CACP-SF compared to PBS (p<0.05) and in HSL compared to both PBS and CACP-SF (p<0.05, p<0.001, respectively). The kinetic coefficient of friction (COF) decreased in CACP-SF compared to PBS (p<0.01) and in HSL compared to PBS (p<0.005). There was no significant difference in kinetic COF between HSL and CACP-SF. The addition of HSL to CACP-SF decreased the static COF from 0.06 to 0.03.

It can accordingly be seen that HSL demonstrated the ability to work as a boundary lubricant and prevent apoptosis in bovine cartilage samples. The chondroprotective properties of the protein are likely linked to anti-adhesive activity and its ability to provide boundary lubrication. CACP-SF samples, being lubricin-free, were unable to provide the same degree of boundary lubrication seen in HSL samples in a cartilage bearing. Due to its ability to re-establish boundary lubrication, in addition to its ability to provide chondroprotection and low coefficient of friction, this study suggests that the use of lubricin in CACP patients may restore chondroprotection. This study also suggest lubricin can be used prophylactically as a chondroprotective agent in joints to prevent chondrocyte apoptosis induced by shear stress on the joint.

### Example 2

### Administration of Lubricin Polypeptide to a Human Experiencing Idiopathic Joint Pain

A patient experiences transient, idiopathic hip and/or knee pain. At the time of evaluation, the pain has resolved and the patient is asymptomatic and shows no clinical signs of cartilage degeneration or damage. The patient has not previously been diagnosed with any condition associated with cartilage degeneration or damage, and in their current asymptomatic state, the patient cannot receive a clinical diagnosis of any such condition.

The physician aspirates the contents of each of the patient's hip and knee joints to evaluate the contents of the synovial fluid. The physician determines that the patient has reduced lubricin concentration and/or lubricin functionality. However, this determination is not sufficient for diagnosis of any clinical condition associated with cartilage degeneration or damage. The patient is then administered lubricin prophylactically to maintain chondrocyte health via intra-articular injection into the capsule of the joint. Lubricin is administered to achieve a bioavailable concentration in the joint capsule of 250-450 μg/mL. Accordingly, between 1-2 mL of fluid containing lubricin in a concentration of 250 μg/mL-10 mg/mL is administered to the patient. The patient is dosed in each joint once per month. Dosage is adjusted in subsequent months based on evaluation of synovial fluid in the joint upon aspiration. Dosing continues once monthly in each knee and hip joint indefinitely to maintain chondrocyte health, thereby preventing onset of any degeneration of cartilage that would lead to clinical symptoms.

### Example 3

### Administration of Lubricin Polypeptide Prophylactically to a Human at Risk of Developing Osteoarthritis

A patient having a family history of osteoarthritis is evaluated and determined to be asymptomatic for osteoarthritis, showing no clinical signs of cartilage degeneration or damage. The physician aspirates the contents of each of the patient's hip and knee joints to evaluate the contents of the synovial fluid. The physician determines that the patient has a minor reduction in lubricin concentration and/or lubricin functionality. However, this determination is not sufficient for diagnosis of any clinical condition associated with cartilage degeneration or damage. The patient is then administered lubricin prophylactically to maintain chondrocyte health via injection into the capsule of the joint. Lubricin is administered to achieve a bioavailable concentration in the joint capsule of 250-450 μg/mL. Accordingly, between 1-2 mL of fluid containing lubricin in a concentration of 250 μg/mL-10 mg/mL is administered to the patient. The patient is dosed in each joint once per month. Dosage is adjusted in subsequent months based on evaluation of synovial fluid in the joint upon aspiration. Dosing continues once monthly in each knee and hip joint indefinitely to maintain chondrocyte health, thereby preventing onset of any degeneration of cartilage that would lead to clinical symptoms of osteoarthritis.

### Example 4

### Administration of Lubricin Polypeptide Prophylactically to an Athlete

A male, aged 50, about to run a marathon who has healthy hip and knee joints and is asymptomatic for any condition related to cartilage degeneration or cartilage trauma is administered lubricin prophylactically 1 hour prior to the event to prevent cartilage damage in the knee and hip incident to joint strain from running A 2 mL solution of lubricin is injected into the joint capsule of each of the left and right knee and the left and right hip at a concentration of 250 μg/mL. The runner runs the marathon and does not experience hip or knee pain or any other sign of trauma to the hip or knee cartilage.

1. A method of maintaining articular cartilage health in a mammal comprising injecting into the capsule of an atraumatic, non-diseased joint of a mammal or a joint of a mammal experiencing transient and/or idiopathic joint pain an amount of a lubricin polypeptide substantially sufficient to prevent stick-slip-induced chondrocyte apoptosis in the superficial zone of articular cartilage and to maintain a lubricin-expressing phenotype of chondrocytes therein.

2. The method of claim 1 comprising injecting an amount of a lubricin polypeptide sufficient to prevent separation of fibronectin and collagenous extracellular cartilage matrix components from chondrocyte surface integrins.

3. The method of claim 1 comprising injecting a recombinant form of a human lubricin polypeptide which contains multiple repeats of an amino acid sequence which is at least 50% identical to KEPAPTT (SEQ ID NO: 2) and comprises O-linked β (1-3) Gal-GalNAc moieties.

4. The method of claim 1 comprising injecting a recombinant form of a human lubricin polypeptide which contains multiple repeats of an amino acid sequence which is at least 50% identical to KEPAPTT (SEQ ID NO: 2) and comprises O-linked β,(1-3)Gal-GalNAc moieties capped with NeuAc.

5. The method of claim 1 comprising injecting an amount of a lubricin polypeptide sufficient to maintain a close packed field of lubricin molecules over weight bearing cartilage surface within the capsule.

6. The method of claim 1, wherein the joint is a knee or hip of a human.

7. The method of claim 1, wherein the amount of lubricin polypeptide is 0.1-2 mL of a solution of 20 μg/mL-10 mg/mL of lubricin.

8. The method of claim 1, wherein the mammal is a non-human mammal.

9. A method of promoting articular cartilage health in a mammal comprising injecting into the capsule of a joint of a mammal previously injected with hyaluronic acid an amount of a lubricin polypeptide substantially sufficient to prevent prospective stick-slip-induced chondrocyte apoptosis in the superficial zone of articular cartilage and to maintain or restore a lubricin-expressing phenotype of chondrocytes therein.

10. The method of claim 9 comprising injecting an amount of a lubricin polypeptide sufficient to prevent separation of fibronectin and collagenous extracellular cartilage matrix components from chondrocyte surface integrins.

11. The method of claim 9 comprising injecting a recombinant form of a human lubricin polypeptide which contains multiple repeats of an amino acid sequence which is at least 50% identical to KEPAPTT (SEQ ID NO: 2) and comprises O-linked β (1-3) Gal-GalNAc moieties.

12. The method of claim 9 comprising injecting a recombinant form of a human lubricin polypeptide which contains multiple repeats of an amino acid sequence which is at least 50% identical to KEPAPTT (SEQ ID NO: 2) and comprises O-linked β,(1-3)Gal-GalNAc moieties capped with NeuAc.

13. The method of claim 9 comprising injecting an amount of a lubricin polypeptide sufficient to maintain a close packed field of lubricin molecules over weight bearing cartilage surface within the capsule.

14. The method of claim 9, wherein the amount of lubricin polypeptide is 0.1-2 mL of a solution of 20 μg/mL-10 mg/mL of lubricin.

15. The method of claim 9, wherein the joint is a knee or hip of a human.

16. The method of claim 9, wherein the mammal is a non-human mammal.


SEQUENCE LISTING (2 sequences)

SEQ ID NO: 1 - protein, 1404 aa, Homo sapiens
MAWKTLPIYLLLLLSVFVIQQVSSQDLSSCAGRCGEGYSRDATCNCDYNCQHYMECCPDFKRVCTAELSCKGRCFESFERGRECDCDAQCKKYDKCCPDYESFCAEVHNPTSPPSSKKAPPPSGASQTIKSTTKRSPKPPNKKKTKKVIESEEITEEHSVSENQESSSSSSSSSSSSTIWKIKSSKNSAANRELQKKLKVKDNKKNRTKKKPTPKPPVVDEAGSGLDNGDFKVTTPDTSTTQHNKVSTSPKITTAKPINPRPSLPPNSDTSKETSLTVNKETTVETKETTTTNKQTSTDGKEKTTSAKETQSIEKTSAKDLAPTSKVLAKPTPKAETTTKGPALTTPKEPTPTTPKEPASTTPKEPTPTTIKSAPTTPKEPAPTTTKSAPTTPKEPAPTTTKEPAPTTPKEPAPTTTKEPAPTTTKSAPTTPKEPAPTTPKKPAPTTPKEPAPTTPKEPTPTTPKEPAPTTKEPAPTTPKEPAPTAPKKPAPTTPKEPAPTTPKEPAPTTTKEPSPTTPKEPAPTTTKSAPTTTKEPAPTTTKSAPTTPKEPSPTTTKEPAPTTPKEPAPTTPKKPAPTTPKEPAPTTPKEPAPTTTKKPAPTAPKEPAPTTPKETAPTTPKKLTPTTPEKLAPTTPEKPAPTTPEELAPTTPEEPTPTTPEEPAPTTPKAAAPNTPKEPAPTTPKEPAPTTPKEPAPTTPKETAPTTPKGTAPTTLKEPAPTTPKKPAPKELAPTTTKEPTSTTSDKPAPTTPKGTAPTTPKEPAPTTPKEPAPTTPKGTAPTTLKEPAPTTPKKPAPKELAPTTTKGPTSTTSDKPAPTTPKETAPTTPKEPAPTTPKKPAPTTPETPPPTTSEVSTPTTTKEPTTIHKSPDESTPELSAEPTPKALENSPKEPGVPTTKTPAATKPEMTTTAKDKTTERDLRTTPETTTAAPKMTKETATTTEKTTESKITATTTQVTSTTTQDTTPFKITTLKTTTLAPKVTTTKKTITTTEIMNKPEETAKPKDRATNSKATTPKPQKPTKAPKKPTSTKKPKTMPRVRKPKTTPTPRKMTSTMPELNPTSRIAEAMLQTTTRPNQTPNSKLVEVNPKSEDAGGAEGETPHMLLRPHVFMPEVTPDMDYLPRVPNQGIIINPMLSDETNICNGKPVDGLTTLRNGTLVAFRGHYFWMLSPFSPPSPARRITEVWGIPSPIDTVFTRCNCEGKTFFFKDSQYWRFTNDIKDAGYPKPIFKGFGGLTGQIVAALSTAKYKNWPESVYFFKRGGSIQQYIYKQEPVQKCPGRRPALNYPVYGEMTQVRRRRFERAIGPSQTHTIRIQYSPARLAYQDKGVLHNEVKVSILWRGLPNVVTSAISLPNIRKPDGYDYYAFSKDQYYNIDVPSRTARAITTRSGQTLSKVWYNCP

SEQ ID NO: 2 - protein, 7 aa, Homo sapiens
KEPAPTT